(19) United States
(12) Patent Application Publication
(10) Pub. No.: US 2004/0096415 A1
(43) Pub. Date: May 20, 2004

(54) Basic, non-aqueous decontaminating fluid

(75) Inventors: Siegfried Franke, Goerlitz (DE); Helmut Stelzmueller, Leutenbach (DE); Hans-Joachim Toepfer, Backnang (DE); Frank Ritscher, Weinstadt (DE)

Correspondence Address: LAW OFFICE OF BARRY R LIPSITZ, 755 MAIN STREET, MONROE, CT, 06468, US

(73) Assignee: Alfred Kaercher GmbH & Co. KG, Winnenden (DE)

(21) Appl. No.: 10/659,045

(22) Filed: Sep. 9, 2003

Related U.S. Application Data
(63) Continuation of application No. PCT/EP02/01095, filed on Feb. 2, 2002.

(30) Foreign Application Priority Data
Mar. 14, 2001 (DE) 101 12 720.0

(51) Int. Cl. A61K 7/075; A61K 7/08

(52) U.S. Cl. 424/70.22

(57) ABSTRACT

In order to provide a basic, non-aqueous decontaminating fluid, such as is used as a universal detoxicant for combating chemical weapons by detoxication of military and civil technical facilities and apparatus, buildings, roads etc., and which can be used more economically but causing no or substantially no attack on painted surfaces, surfaces coated with plastics/elastomers, particularly surfaces of metals, plastics materials, or elastomers when treated for the necessary period of treatment, the invention proposes that said basic, non-aqueous decontaminating solution contains

20 to 40 wt % of an aliphatic C₂₋₆ alcohol;

20 to 40 wt % of an aliphatic C₂₋₆ amino alcohol;

20 to 50 wt % of a cyclic C₂₋₅ acid amide and/or an aliphatic C₂₋₆ diamine; and

0.5 to 2.6 mol/L of an alkali metal alkoxide (alkali metal alcoholate, alkali metal alkanolate) and/or an alkali metal aminoalkanoxide (alkali metal aminoalcoholate, alkali metal aminoalkanolate).

The present disclosure relates to the subject matter disclosed in international application No. PCT/EP02/01095 of Feb. 2, 2002, which is incorporated herein by reference in its entirety and for all purposes.

### BACKGROUND OF THE INVENTION

The invention relates to a basic, non-aqueous decontaminating fluid, such as is used as a universal detoxicant for combating chemical weapons by detoxication of military and civil industrial facilities and apparatus, buildings, roads etc.

Examples of such basic, non-aqueous decontaminating fluids are disclosed in U.S. Pat. No. 3,079,346 or German patent specification DD 299 458 A7. Their basicity is due to the alkali metal alkoxides (alkali metal alcoholates, alkali metal alkanolates) present therein and their anions. These are the main reactive components for conversion of toxic contaminants which may be used in military and terrorist activities, such as chemical weapons in the group comprising the dichlorodiethyl sulfides (such as mustard gas), the organofluorophosphoryls (such as soman) or the organothiocholinephosphoryls (such as VX).

A drawback of the known decontaminating fluids is, on the one hand, their adverse effect on plastics materials, elastomers, and paint coatings, which are caused to swell or soften and come off the substrate and also lead to corrosive phenomena on metals and metal alloys.

### SUMMARY OF THE INVENTION

It is an object of the present invention to propose a decontaminating fluid of the aforementioned type, which is on the one hand more economical in use and shows, on the other hand, no or substantially no tendency to attack painted surfaces, surfaces coated with plastics/elastomers, especially surfaces of metals, plastics materials, and elastomers, during the requisite periods of treatment.

According to the invention, this object is achieved using the aforementioned basic, non-aqueous decontaminating fluid when a formulation thereof contains from 0.5 to 2.6 mol/L of alkali metal alkoxide (alkali metal alcoholate, alkali metal alkanolate) and/or of alkali metal aminoalkoxide (alkali metal aminoalcoholate, alkali metal aminoalkanolate). This formulation contains:

20 to 40 wt % of an aliphatic C₂₋₆ alcohol;

20 to 40 wt % of an aliphatic C₂₋₆ amino alcohol, and

20 to 50 wt % of a cyclic C₂₋₅ acid amide and/or of an aliphatic C₂₋₅ diamine.

To achieve the same decontaminating effect with the aforementioned conventional decontaminating fluids it is necessary to use larger quantities thereof per unit area, and some of them require a longer periods of action.

The decontaminating fluid of the invention contains a chemical equilibrium system consisting of aliphatic alcohols and amino alcohols, their alkali metal alkoxides and their ions.

The decontaminating fluid of the invention can be used in suitable conventional military and civil decontaminating devices for decontaminating military technology, aircraft, ships, and rail vehicles; facilities, such as buildings, roads, and runways; and also laboratory apparatus.

The non-aqueous decontaminating fluid of the invention is, on account of its stronger basicity, more reactive than the prior decontaminating fluids. That is the reason for its greater effectiveness and the lower rate of application thereof required per unit area to be decontaminated.

The increase in reactivity makes it possible to shorten the period of action, so that damage to contaminated surfaces comprising plastics materials, elastomers, paint coatings etc. can be avoided. Corrosive phenomena on metals and metal alloys do not occur, not even when the fluid is allowed to act for a relatively long period.

Furthermore, the fluid of the invention is less hygroscopic, which means that application thereof, even under conditions of high air humidity, leads to no formation of mist.

The decontaminating fluid disclosed in U.S. Pat. No. 3,079,346 has a strong hygroscopic action, so that the occurrence of mist is possible even under conditions of low air humidity. This will certainly not happen with the decontaminating fluid of the invention.

The decontaminating fluid of the invention can be rinsed off from the treated surfaces with water, which must not necessarily be applied under high pressure. Due to the fact that smaller quantities of decontaminating fluid have to be applied per unit area, the consumption of water used for rinsing is also lower.

The decontaminating fluid disclosed in DD 299 458 A7 is less basic than the decontaminating fluid of the invention and in addition contains N-methyl-ε-caprolactam, which is less economical.

Another advantageous of the present invention is that the fluid can be used in a great variety of ways, ie in both large and small devices, and also by primitive means, if necessary compounded in such means.

Preferred decontaminating fluids of the invention contain at least one alkali metal alkoxide and one alkali metal aminoalkoxide, which are produced by the introduction (addition) of the corresponding pure alkali metal or an alkali metal hydroxide. They can alternatively be introduced in the form of commercially available alkali metal alkoxides.

Other preferred decontaminating fluids contain, as aliphatic alcohols, propanols and/or butanols.

A preferred aminoalcohol component used is 2-amino-1-butanol, N,N-dimethylaminoethanol, and/or N-methyldiisopropanolamine.

A preferred acid amide for use in the recipe of the basic, non-aqueous decontaminating fluid of the invention is N-methyl-2-pyrrolidone.

In order to improve the application properties, there can be added to the decontaminating fluid one or more co-solvents, such as up to 10 wt % of diols and/or up to 20 wt % of a liquid aliphatic or aromatic hydrocarbon.

Finally, the invention relates to a process for decontaminating surfaces, particularly painted or polymer-coated surfaces or plastics or elastomer surfaces, which process is characterized by the use of the decontaminating fluid of the invention defined above.

In the present invention, the decontaminating fluid as described above is applied at rates of from 0.05 to 0.2 L/m² surface area depending on the type contaminant and the estimated density of contamination. Such small amounts attain excellent decontamination results even when combating chemically relatively stable, highly poisonous warfare agents at densities of contamination of 10 g per m², and even when the warfare agents are present in a thickened form and the duration of action thereof has been several hours.

### DETAILED DESCRIPTION OF THE INVENTION

These and other advantages of the invention are illustrated in greater detail below with reference to examples.

### Examples of Formulations

| Component | Formulation A | | Formulation B | | Formulation C | |
|---|---|---|---|---|---|---|
| aliphatic | 2-amino-1-buta- | 30 | N-methyldiiso- | 30 | N,N-dimethyl- | 30 |
| aminoalcohol [wt %] | nol | | propanolamine | | aminoethanol | |
| acid amide/ | N-methylpyrroli- | 40 | 1.3-diaminopropane | 40 | N-methylpyrrolidone | 40 |
| amine [wt %] | done | | | | | |
| aliphatic | n-butanol | 30 | Isopropanol | 30 | tert-butanol | 30 |
| alcohol [wt %] | | | | | | |
| total content of | | 1 | | 1 | | 1 |
| alcoholates in mol/L | | | | | | |

### EXAMPLES

In the following examples, alkyd resin-coated metal plates were contaminated in field experiments over a period of 3 hours with the relevant chemical weapons, prior to decontamination, such that the warfare agents could penetrate into the layers of alkyd paint. The plates were set up at different angles of inclination. In all of the decontamination experiments described in the examples, the ambient temperature was from 10° to 15° C.

### Example 1

Following the duration of action, the average density of contamination caused by the warfare agent mustard gas (HD) on the alkyd resin-coated metal plates was still 10,840 mg/m².

Irrespective of their degrees of inclination, the plates were sprayed with 0.1 L/m² of the decontaminating fluid of the invention according to formulation A. The fluid was allowed to act for 5 minutes, after which the plates were treated with warm water (ca 80° C.) to rinse off the decontaminating fluid.

Analysis carried out directly after gave an average residual contamination by mustard gas of 55 mg/m². This corresponds to a decontamination success rate of 99.5%.

### Example 2

In a manner similar to that described in Example 1 plates were contaminated with the chemical weapon VX. Following the period of action, the density of contamination was 8030 mg VX/m².

Treatment was again carried out with 0.1 L/m² of formulation A. Following a period of action of 5 minutes and aftertreatment with water (ca 80° C.), an average residual contamination of 3.6 mg VX/m² was found. This corresponds to a decontamination success rate of 99.96%.

### Example 3

In a manner similar to that described in Example 1 plates were contaminated with the chemical weapon soman (GD). Following the period of action, the average density of contamination was 4160 mg GD/m².

Treatment was again carried out with 0.1 L/m² of formulation A. After the period of action of 5 minutes and the aftertreatment with water (ca 80° C.), the residual contamination was on average 98.8 mg GD/m². This corresponds to a decontamination success rate of 97.53%.

### Examples 4 to 6

Examples 1 to 3 were again carried out in a similar manner but using the decontaminating fluid of formulation B. The decontaminating effect on these 3 chemical weapons was substantially the same as with formulation A.

### Examples 7 to 9

Examples 1 to 3 were again carried out in a similar manner but using the decontaminating fluid of formulation C. The decontaminating effect was, viewed generally, somewhat lower and was on average 98.3%, the decontamination success rate being on average 99.4 on mustard gas and 99.8% on VX.

The results of these field experiments show that periods of treatment of 5 minutes using the decontaminating fluids of the invention achieve residual densities of decontamination which are substantially lower than the limits declared as permissible.

These very good decontamination success rates could be confirmed for an identical procedure for decontamination of plates contaminated with thickened chemical weapons.

The metal plates coated with alkyd resin and treated with the decontaminating fluids according to formulations A, B and C were inspected after decontamination, and no damage to the alkyl resin coating could be found in any of Examples 1 to 9.

1. A basic, non-aqueous decontaminating fluid, comprising
20 to 40 wt % of an aliphatic C₂₋₆ alcohol;
20 to 40 wt % of an aliphatic C₂₋₆ amino alcohol;
20 to 50 wt % of a cyclic C₂₋₅ acid amide and/or an aliphatic C₂₋₆ diamine; and
0.5 to 2.6 mol/L of an alkali metal alkoxide (alkali metal alcoholate, alkali metal alkanolate) and/or an alkali metal aminoalkanoxide (alkali metal aminoalcoholate, alkali metal aminoalkanolate).

2. The decontaminating fluid as defined in claim 1, wherein the alkali metal alkoxide is selected from C₁₋₆ alkoxides.

3. The decontaminating fluid as defined in claim 1, wherein the alkali metal alkoxide(s) (alcoholate(s), alkanolate(s)) and the ions thereof are formed by introducing the corresponding pure alkali metal or an alkali metal hydroxide or a commercial alkali metal alkoxide to the solvent components.

4. The decontaminating fluid as defined in claim 1, wherein the aliphatic alcohol present is a propanol and/or butanol.

5. The decontaminating fluid as defined in claim 1, wherein the amino alcohol present is 2-amino-1-butanol, N,N-dimethylaminoethanol, and/or N-methyldiisopropanolamine.

6. The decontaminating fluid as defined in claim 1, wherein the cyclic acid amide present is N-methyl-2-pyrrolidone.

7. The decontaminating fluid as defined in claim 1, wherein the fluid also contains, as co-solvents,
up to 10 wt % of a diol and/or
up to 20 wt % of an aliphatic or aromatic liquid hydrocarbon.

8. A process for the decontamination of surfaces, particularly painted, polymer-coated or plastics surfaces, characterized by the application of a decontaminating fluid as defined in claim 1.

9. The process as defined in claim 8, wherein the decontaminating fluid is applied, particularly by spraying, at a rate of from 0.05 to 0.2 L/m² surface area.

10. The process as defined in claim 8, wherein the decontaminating fluid is allowed to act for from 5 to 15 min and then rinsed off.

11. The process as defined in claim 8, wherein the surfaces that have been treated with the decontaminating fluid are treated with cold, warm, or hot water or with superheated steam such that the decontaminating fluid and the reaction products are rinsed off.
